(19) United States
(12) Patent Application Publication
(10) Pub. No.: US 2007/0098662 A1
(43) Pub. Date: May 3, 2007

(54) Cosmetic composition promoting oxygen transport into the skin

(75) Inventors: Gabriele Blume, Strasse (DE); Michael Sacher, Schluchtern (DE); Dirk Teichmuller, Schluchtern (DE)

Correspondence Address: SIMPSON & SIMPSON, PLLC, 5555 MAIN STREET, WILLIAMSVILLE, NY, 14221-5406, US

(73) Assignee: ROVI GMBH & CO., KOSMETISCHE ROHSTOFFE KG, SCHLUCHTERN GERMANY (DE)

(21) Appl. No.: 10/567,631

(22) PCT Filed: Aug. 4, 2004
(86) PCT No.: PCT/EP04/51702

§ 371 Date: Dec. 22, 2006

(30) Foreign Application Priority Data
Aug. 11, 2003 (DE) 103-36-841.8

(51) Int. Cl. A61K 8/73

(52) U.S. Cl. 424/70.13; 424/70.31

(57) ABSTRACT

A cosmetic composition containing from about 1% to about 50% by weight of membrane-forming sphingolipids and/or galactolipids and about 5% to about 50% by weight of a fluorocarbon or fluorocarbon mixture charged with oxygen. Surprisingly, it has been shown that the use of membrane-forming sphingolipids and/or galactolipids as transport vesicles or for the formation of transport vesicles for a fluorocarbon or fluorocarbon mixture charged with oxygen results in excellent transport of oxygen through the stratum corneum of the skin and the epidermis in a manner which is far superior to known transport systems.

### BACKGROUND OF THE INVENTION

The invention relates to a cosmetic composition to assist the transport of oxygen into the skin with vesicles as carriers for oxygen transport.

The term “cosmetic” composition as used in the present invention also encompasses “pharmaceutical” compositions, i.e. compositions which fall within the ambit of the pharmaceutical laws.

Molecular oxygen is important in making energy available in many processes which occur in the cells of higher organisms. After taking up oxygen, for example via the lungs, oxygen is bound to erythrocytes and transported to their target sites via arterial blood vessels and small capillaries. In that location, the oxygen is released to the tissue and transferred into the mitochondria via the respiratory path with the release of energy. Oxygen is also taken in via the skin (transcutaneously) and transported to the underlying tissues. In humans, it has been observed that from the age of about 20 years, the transcutaneous oxygen pressure frequently reduces. This local reduction in oxygen pressure goes hand in hand with reduced exchange between water in the blood plasma and the extracellular fluid—subcutaneous tissue loses moisture and contains smaller amounts of nutrients. The diffusion capacity of the capillaries drops off in these regions.

Such an oxygen deficiency can result in the skin, in particular that of the face, losing its youthful and healthy appearance. This is termed premature ageing of the facial skin, which is accompanied by increased wrinkle formation. The cosmetics industry offers a large number of preparations which are meant to counter such premature skin ageing and wrinkle formation. Some of those preparations are supposed to transport molecular oxygen into the subcutaneous tissue along with having a moisturizing effect, to thereby balance the increasing lack of supply of such tissue with oxygen with increasing age. The various preparations which are available have varying degrees of effectiveness.

The use of fluorocarbons as oxygen binding compounds in cosmetic and medical preparations is known. U.S. Pat. No. 4,366,169 describes the use of fluorocarbons for the treatment of skin lesions and wounds, in particular bums, wherein the oxygen-containing fluorocarbon is applied to the skin either directly or as an emulsion, or on suitable strips or the like. U.S. Pat. No. 4,569,784 describes the production of a gel with gas transport properties for use on the skin. In the process, an organic liquid which is not miscible with water, for example a fluorocarbon, is emulsified in the presence of an emulsifying agent. A concentration process is then carried out which results in the formation of a gel phase. In the next step, the clear fluid is separated from the pasty solid (gel phase) by decanting, filtration or evaporation. The gel is used in suitable formulations on the skin and works without, however, penetrating the stratum corneum of the skin. European patent application EP-A-0 296 661 describes a single phase system containing a fluorocarbon, which works as an isotropic or anisotropic formulation in cosmetics and also as a dermaticum as an oxygen transporter. In that patent, fluorocarbons are emulsified with a maximum concentration of 50% in water with perfluorinated emulsifying agents of the alkanesulphonic acid amide type in the presence of an aliphatic alcohol as an additional emulsifying agent. International patent application WO-A-8908459 describes a perfluorocarbon emulsion with phospholipid vesicles as a blood replacement, wherein phospholipid monomers are polymerized. WO-A-9100110 discloses fluorocarbon emulsions with phospholipids wherein the phospholipid has saturated carbon bonds. WO-A-9206676 discloses oil-filled vesicles formed from phospholipids the structure of which corresponds to the usual structure of vesicles. EP-A-0 647 131 describes a dermaticum for transporting oxygen into skin, which contains asymmetric lamellar aggregates constituted by phospholipids with a phosphatidylcholine content of 30-99% by weight and a fluorocarbon or fluorocarbon mixture charged with oxygen, wherein the aggregates have a skin penetration which depends on the critical solubility temperature in n-hexane of the selected fluorocarbon or fluorocarbon mixture.

The majority of known compositions, when intended for the transport of molecular oxygen into the skin, suffer from the disadvantage that they are not capable of passing through the stratum corneum of the skin and epidermis and transporting molecular oxygen into the tissues bordering them in sufficient quantities.

### BRIEF DESCRIPTION OF THE INVENTION

The present invention aims to overcome the disadvantages of prior art compositions and to provide a cosmetic composition which assists the transport of molecular oxygen into the skin through the stratum corneum and the epidermis into the bordering tissues thereof and thus to increase oxygen concentrations in the tissue and activate metabolic processes.

This aim is achieved in accordance with the invention, wherein a cosmetic composition of the type described above contains 1% to 50% by weight of membrane-forming sphingolipids and/or galactolipids and 5% to 50% by weight of a fluorocarbon or fluorocarbon mixture charged with oxygen.

Surprisingly, it has been shown that the use of membrane-forming sphingolipids and/or galactolipids as transport vesicles or for the formation of transport vesicles for a fluorocarbon or fluorocarbon mixture charged with oxygen results in excellent transport of oxygen through the stratum corneum of the skin and the epidermis in a manner which is far superior to known transport systems.

### DETAILED DESCRIPTION OF THE INVENTION

Sphingolipids are complex lipids with sphingosine or a similar base as the basic structure. They are important components of plant and animal cell membranes and contain three characteristic components: one molecule of fatty acid, one molecule of sphingosine or sphingosine derivative and a polar (head) group, which can sometimes be very large and complex. Sphingosine is one of about 30 long chain aminoalcohols which are found in different types of sphingolipids. In mammals, sphingosine (4-sphingenine) and dihydrosphingosine (sphinganine) are the most usual bases of sphingolipids; in higher plants and yeasts, it is phytosphingosine (4-hydroxysphinganine) and in marine invertebrates, they are doubly unsaturated bases such as 4,8-sphingadins. The sphingosine base is bonded to a long chain unsaturated or monounsaturated fatty acid containing 18 to 26 carbon atoms via an amide bond in its amino group. This compound, which contains two non polar chains, is ceramide, the characteristic basic structure of all sphingolipids. Unless expressly otherwise given, in the context of this application the term “ceramide” will have the same meaning as the term “sphingolipid”.

The various derivatives of sphingolipids or ceramides can be divided further into groups depending on their behaviour and structural chemical features. The most usual sphingolipids in the tissues of higher animals are sphingomyelins, which contain phosphorylethanolamine or phosphorylcholine as their polar groups. They are zwitterions at pH 7.

A second group of sphingolipids contains one or more neutral sugars as the polar group; they thus have no electric charge and hence are termed neutral glycosphingolipids. Cerebrosides are the simplest members of this group; they have only one monosaccharide in β-glycosidic combination with their hydroxyl group as the polar group. D-galactose is present in the cerebrosides of the brain and nervous system; they are thus termed galactocerebrosides. Cerebrosides are also present in small amounts in non neutral tissues; here, they contain mainly D-glucose and hence are termed glucocerebrosides. Sulphate esters of galactocerbrosides are termed sulphatides, which are also present in brain tissue. Cerebrosides and sulphatides contain fatty acids containing 22 to 26 carbon atoms. A common fatty acid in cerebrosides is cerebronic acid.

Neutral glycosphingolipids with one disaccharide are termed dihexosides. Tri- and tetra-hexosides are also known. The sugars in these glycosphingolipids are D-glucose, D-galactose, N-acetyl-D-glucosamine and N-acetyl-D-galactosamine. Neutral glycosphingolipids are important components of cell surfaces in animal tissues. Their non polar portion is anchored in the double lipid layer of the membrane, while the polar portion projects from the surface.

A third group of sphingolipids is acidic glycosphingolipids, which are termed gangliosides. Their oligosaccharide portion contains one or more sialinic acids. Gangliosides are present in grey matter.

Galactolipids are membrane lipids primarily present in plants. MGDG (monogalactosyldiacylglycerol) and DGDG (digalactosyldiacylglycerol) are the most common galactolipids in higher plants. They are primarily present in plastides and accumulate in particular in the thyalkoids of chloroplasts.

The vesicle of the invention differs from liposomes formed from phospholipids containing large quantities of phosphatidylcholine which are also used in cosmetics. Phospholipids are cell membrane components. Liposomes formed from phospholipids are thus regularly used in cosmetics as vesicles to transport various active ingredients into cells. It has, however, surprisingly been discovered that the vesicles employed in the composition of the invention formed from sphingolipids and/or galactolipids are more suitable for transporting active ingredients through the skin than known liposomes formed from phospholipids. In particular, they allow effective transport through the stratum corneum and into deep layers of the skin, better than that with known liposomes. Since the lipids of the invention are either naturally present or closely resemble lipids which are found in the stratum corneum, applying the cosmetic composition of the invention to the skin simultaneously stabilizes the natural skin barrier of the stratum corneum.

In comparison with phospholipid vesicles, the composition of the invention has both increased oxygen transport to the desired location of action and a much better tolerance by the skin.

The action of the fluorocarbon-containing composition of the invention resides in the release of oxygen depleted tissue on topical application. It is also applicable to fat tissue which is deficient in oxygen, and also for arteriosclerotic deficiency. As will be explained in more detail below, the composition of the invention is also suitable for delivering oxygen to diabetic legs and smoker's legs. These diseases involve peripheral blockages which result in a deficiency of blood and oxygen in the tissue.

The composition of the invention is formulated into salves, creams, lotions and other aqueous or alcoholic dermatological administration forms depending on its intended use, wherein the fluorocarbon content and thereby the oxygen availability can be varied within wide limits. The fluorocarbons may be partially charged or saturated with oxygen-forming gas prior to incorporation into a dermatological system such as a gel, paste, powder, salve, cream or lotion. Even saturation with oxygen from atmospheric air by establishing the usual equilibrium provides a higher oxygen capacity than any known comparable system. The composition of the invention can also be placed on strips, sticking plasters, dressings and other means that come into contact with the skin. As an example, it may also be applied as a spray.

The term “fluorocarbon” as used here means perfluorinated or highly fluorinated hydrocarbon compounds or mixtures which are capable of transporting oxygen. Highly fluorinated hydrocarbon compounds are, within the context of the present invention, those in which the majority of hydrogen atoms are replaced by fluorine atoms, such as bis-F-(alkyl)ethene, which unfortunately are known to be chemically and biologically inert and thus non toxic. This is mainly achieved if up to 90% of the hydrogen atoms are replaced by fluorine atoms. Preferably, in the present invention, fluorocarbons are used in which at least 95% of the hydrogen atoms have been replaced, preferably 98% and more preferably 100%. Individual fluorine atoms may also be replaced by other halogen atoms such as bromine or chlorine.

Many oxygen-binding fluorocarbons are suitable for use in the cosmetic composition of the invention. Examples of suitable fluorocarbons are aliphatic straight chain and branched fluoroalkanes, mono or bi-cyclic and also fluoroalkyl-substituted fluorocycloalkanes, and perfluorinated aliphatic or bicyclic amines, bis-(perfluoroalkyl)-ethenes or mixtures thereof. Particularly preferred compounds are perfluorodecalin, F-butyltetrahydrofuran, perfluorotributylamine, perfluorooctylbromide, bis-fluoro-(butyl)-ethene and bis-fluoro-(hexyl)-ethene or C₆-C₉-perfluoroalkanes. A particularly preferred compound is perfluorodecalin. The fluorocarbon or fluorocarbon mixtures as used in the invention are employed in amounts of 5-50% by weight, preferably 10-50% by weight, more preferably 15-25% by weight with respect to the total weight of the cosmetic composition.

Regarding the effect of the transport vesicle for oxygen transport into the skin of the invention compared with phospholipid liposomes, the inventors of the present application assume the following mechanism which, however, is not in any way limiting as regards the scope of the application: the sphingolipid and/or galactolipid vesicles of the composition of the invention initially pass into the upper layers of the skin, where they then merge with this skin layer, in particular the stratum corneum, and release the active ingredient. The vesicles are occlusive, i.e. they build a barrier against the reverse transport of the released active ingredient or oxygen. The oxygen distributes itself or diffuses further into deeper layers beneath the stratum corneum, where it can then have its advantageous effects. In contrast, phospholipid liposomes remain substantially stable and penetrate or pass into the skin as the vesicle. Due to their greater stability, less active ingredient is released from the liposomes than with the vesicles of the invention, which also explains the improved efficacy of the vesicles of the invention.

In a preferred implementation of the cosmetic composition of the invention, the sphingolipids or ceramides are selected from neutral glycosphingolipids, such as cerebrosides, galactocerebrosides, glucocerebrosides and sulphatides.

In a further preferred implementation of the cosmetic composition of the invention, the galactolipids are selected from monogalactosyldiacylglycerol and digalactosyldiacylglycerol.

In a further preferred implementation of the cosmetic composition of the invention, the vesicles are contained in the composition as vesicles with a double lipid layer membrane.

The cosmetic composition of the invention can advantageously contain other dermatologically acceptable additives, emulsifying agents, preservatives, excipients, solublizing agents, thickening agents and/or stabilizers.

Particularly preferably, the cosmetic composition of the invention further contains 5-20% by weight of oils and/or waxes, preferably vegetable oils and/or vegetable waxes. Jojoba oil, which has a particularly pleasurable skin feel on application of the cosmetic composition, is particularly preferred. Sunflower seed oil is also suitable. Advantageously, the sphingolipids and/or galactolipids are in the form of a solution in oil in the cosmetic composition of the invention. Such an oil solution preferably contains 10-20% by weight of sphingolipids and/or galactolipids in oil.

In accordance with a further preferred implementation, the cosmetic composition contains 10-50% by weight of alcohol, preferably glycerin, ethanol and/or glycols such as 1,2-pentyleneglycol, 1,3-butyleneglycol or 1,2-pentyleneglycol. Glycerin is preferably in the cosmetic composition of the invention in an amount of 10-20% by weight, more preferably 14-18% by weight. Glycerin acts as a moisturizer for the skin and stabilizes the composition of the invention. Ethanol and/or 1,2-pentyleneglycol, 1,3-butyleneglycol or 1,2-pentyleneglycol are preferably present in an amount of 5-30% by weight, particularly preferably 15-25% by weight. Glycols act as moisturizers and solubilizing agents. Further, they have an antimicrobial spectrum of action.

In a further preferred implementation, the cosmetic composition of the invention contains 0.5% to 5% by weight, particularly preferably 1.0% to 3.0% by weight of a polyethylene glycol fatty acid glyceride, preferably a fatty acid glyceride of polyethylene glycol 25 to polyethylene glycol 75. Particularly preferably, the fatty acid glyceride is a shea butter glyceride. Alternatively, coconut glyceride or other oil glycerides are suitable. The polyethylene glycol fatty acid glycerides provide steric protection for the transport vesicle in the composition of the invention and thus stabilize the vesicle suspension.

Depending on the composition, it may be advantageous or necessary to add preservatives and/or thickening agents to the cosmetic composition of the invention. Preservatives may be added in an amount of 0.01% to 1% by weight; thickening agents may be added in an amount of 0.05% to 2% by weight. Particularly preferably, however, preservative-free compositions are used.

In a further implementation of the cosmetic or pharmaceutical composition of the present invention, the composition further contains a natural or synthetic capsaicin (nonylic acid vanillylamide), preferably in an amount of 0.1% to 1% by weight, more preferably in an amount of 0.2% to 0.6% by weight, and/or nicotinic acid and/or nicotinamide and/or nicotinic acid ester, preferably in an amount of 0.5% to 5% by weight, particularly preferably in an amount of 0.5% to 3% by weight. Because of the analgesic and haemorrhagic effect of the above substances, this implementation of the composition of the invention can advantageously be used to regenerate and improve the condition of diabetic legs and smoker's legs. In diabetic leg and smoker's leg, the oxygen supply is severely restricted or perturbed compared with the normal state. Using the composition of the invention can allows this oxygen deficiency to be at least partially regained and the accompanying pain can be alleviated. Simultaneous delivery of the above substances of this implementation of the invention leads to the production of heat, which encourages regeneration.

The invention will now be described in more detail with the aid of the following examples.

### EXAMPLE 1

A particularly preferred cosmetic composition of the invention for skin treatment contains:

| | |
|---|---|
| 20.0% by weight | 1,2-propylene glycol |
| 16.0% by weight | Glycerin |
| 10.0% by weight | Sphingolipid-oil/wax solution (15% by weight |
| | glycosphingolipid in jojoba oil) |
| 2.0% by weight | Polyethylene glycol 75 shear butter glyceride |
| 0.2% by weight | Xanthan gum |
| 20.0% by weight | Perfluorodecalin |
| 0.05% by weight | Preservative (EuxylK702 ®, Schülke & Mayr, |
| | DE) |
| Qs 100% by weight | water |

To prepare this composition, 1,2-propylene glycol and glycerin were carefully mixed to homogeneity and placed in a beaker (clear, colourless, slightly viscous solution). The sphingolipid-oil/wax solution, the polyethylene glycol75 shea butter glyceride and xanthan gum were incorporated using a Turrax homogenizer (homogenization at 10000 rpm). The viscous, pale beige solution was then homogenized for a further 20 minutes. With further constant Turrax homogenization (ca. 10000 rpm), the perfluorodecalin was incorporated and homogenization was continued until a white homogeneous emulsion was obtained. Water was added with further Turrax homogenization (ca. 10000 rpm). The white solution was then further homogenized for 20 minutes and stored. The pH of the emulsion was adjusted to a pH of 4.5 to 6.5 using sodium hydroxide. The vesicle size measured in the cosmetic composition was 150 to 300 nm.

### EXAMPLE 2

A further composition for diabetics and smoker's legs contained:

| | |
|---|---|
| 20.0% by weight | 1,2-propylene glycol |
| 16.0% by weight | Glycerin |
| 10.0% by weight | Sphingolipid-oil/wax solution (15% by weight |
| | glycosphingolipids in sunflower seed oil) |
| 2.0% by weight | Polyethylene glycol 75 shear butter glyceride |
| 0.2% by weight | Xanthan gum |
| 0.5% by weight | Nonylic acid vanillylamide |
| 20.0% by weight | Perfluorodecalin |
| 0.05% by weight | Preservative (EuxylK702 ®, Schülke & Mayr, |
| | DE) |
| Qs 100% by weight | water |

To prepare this composition, 1,2-propylene glycol and glycerin were carefully mixed to homogeneity and placed in a beaker. The nonylic acid vanillylamide was completely dissolved in this mixture (clear, almost colourless, slightly viscous solution). The sphingolipid-oil/wax solution, the polyethylene glycol 75 shea butter glyceride and xanthan gum were incorporated using a Turrax homogenizer (homogenization at 10000 rpm). The viscose, pale beige solution was then homogenized for a further 20 minutes. With further constant Turrax homogenization (ca. 10000 rpm), the perfluorodecalin was incorporated and homogenization was continued until a white homogeneous emulsion was obtained. Water was added with further Turrax homogenization (ca. 10000 rpm). The white solution was then further homogenized for 20 minutes and stored. The pH of the emulsion was adjusted to a pH of 4.5 to 6.5 using sodium hydroxide. The vesicle size measured in the cosmetic composition was 200 to 450 nm.

### EXAMPLE 3

The efficacy of the cosmetic composition of the invention of Example 1 was tested as regards oxygen transport through the skin of six healthy female volunteers aged between 20 and 50. An earlier study by C Artmann et al (SÖFW Journal 15, 1993, pp 6-8), which determined the partial pressure of oxygen (pO₂) in 361 volunteers, showed that male volunteers had much lower pO₂ values than female volunteers. In the present experiment, measurements of the transcutaneous oxygen partial pressure (pO₂) were carried out on the inside of the forearm with a polarographic probe (Clark method). The transcutaneous partial pressures of oxygen were recorded as “mm Hg” using an OMED (pO₂) analyzer (Bretzfeld, Germany). To produce local arterialization, the probe was heated to a constant temperature of 42° C. (slightly hyperthermal), allowing the probe to make a quantitative determination of the partial pressure of oxygen in the region of the arterialized skin tissue. The starting value for the partial pressure of oxygen without treatment with the cosmetic composition of the invention was measured after 20 minutes. Then 5 μl of the cosmetic composition of the invention of Example 1 was applied to a 1 cm² region of skin on one arm of each volunteer and the other arm was left untreated as the control. The experiment was commenced at 9.00 am and over a period of six hours, the change in the partial pressure of oxygen was measured at intervals of 1.5 hours. The results are shown in Table 1 below.

The data shows that the transcutaneous oxygen pressure is highly dependent on the time of day. While very low values were observed for the partial pressure of oxygen in all volunteers in the middle of the day, in the afternoon, the partial pressure of oxygen climbed again in all volunteers. The results shown in Table 1, which provide average values for the measurements in all six volunteers, show a clear increase in oxygen concentration in the deeper layers of the skin even after 1.5 hours. The significant increase in the oxygen content in the parts of the skin treated with the composition of the invention compared with the untreated skin was substantially the same over the period of six hours of the experiment. The difference in the partial pressure of oxygen between treated and untreated skin was about 9 mm Hg on average, which corresponds to a value which can be achieved by inhaling pure oxygen. A single application of the cosmetic composition of the invention, then, can produce a substantial increase in the oxygen concentration in the skin.

TABLE 1

| | | Untreated pO₂ | Treated pO₂ |
|---|---|---|---|
| Time [h] | Time of day | [mm Hg] | [mm Hg] |
| 0 | 9:00 | 86 | 86 |
| 1.5 | 10.30 | 83 | 91 |
| 3 | 12:00 | 79 | 88 |
| 4.5 | 13:30 | 72 | 81 |
| 6 | 15:00 | 77 | 86 |

What is claimed is:

1-12. (canceled)

13. A cosmetic composition to assist oxygen transport into the skin with vesicles as carriers, wherein the composition comprises 1% to 50% by weight of lipid in the form of membrane-forming lipid conjugate selected from the group consisting of sphingolipids, galactolipids and mixtures thereof, and 5% to 50% by weight of a fluorocarbon or mixture of fluorocarbons charged with oxygen.

14. A cosmetic composition according to claim 1, wherein the sphingolipids are selected from neutral glycosphingolipids.

15. A composition according to claim 14 wherein the neutral glycosphingolipids are selected from the group consisting of cerebrosides, galactocerebrosides, glucocerebrosides and sulphatides.

16. A cosmetic composition according to claim 13 wherein the galactolipids are selected from the group consisting of monogalactosyldiacylglycerol and digalactosyldiacylglycerol.

17. A cosmetic composition according to claim 13 wherein the vesicles comprise membrane-forming lipid conjugate selected from the group consisting of sphingolipids, galactolipids and mixtures thereof and vesicles contained in the composition have a double lipid layer membrane.

18. A cosmetic composition according to claim 13 wherein the vesicles in the composition are present as nanoparticles or a nanoemulsion.

19. A cosmetic composition according to claim 18 wherein the vesicles have a single lipid layer membrane.

20. A cosmetic composition according to claim 13 wherein the fluorocarbon is perfluorodecalin.

21. A cosmetic composition according to claim 13 wherein the composition further contain 5% to 20% by weight of lipid conjugate solvent selected from oils, waxes or mixtures thereof.

22. A cosmetic composition according to claim 21 where the oil, wax or mixtures thereof comprise vegetable oils, vegetable waxes and mixtures thereof.

23. A cosmetic composition according to claim 21 where the lipid conjugate solvent comprises jojoba oil.

24. A cosmetic composition according to claim 13 wherein the composition further contains a capsaicin in an amount of 0.1% to 1% by weight.

25. A composition according to claim 24 where the capsaicin is present in an amount of 0.2% to 0.6% by weight.

26. A composition according to claim 25 further containing a nicotinic compound selected from the group consisting of nicotinic acid, nicotinamide, nicotinic acid ester, and mixtures thereof in an amount of 0.5% to 5% by weight.

27. A composition according to claim 26 where the nicotinic compound is present in an amount of 0.5% to 3% by weight.

28. A cosmetic composition according to claim 13 where the composition further contains 10% to 50% by weight of an alcohol.

29. A cosmetic composition according to claim 28 where the alcohol is selected from the group consisting of glycerin, ethanol and glycols.

30. A cosmetic composition according to claim 29 where the alcohol is a glycol selected from the group consisting of 1,2-propylene glycol, 1,3-butylene glycol, 1,2-pentylene glycol.

31. A cosmetic composition according to claim 13 wherein the composition further contains 0.5% to 5% by weight of a polyethylene glycol fatty acid glyceride.

32. A cosmetic composition according to claim 13 where the composition further contains at least one additive selected from preservatives in an amount of 0.01% to 1.0% by weight and thickening agents in an amount of 1.0% to 2.0% by weight.

33. A cosmetic composition according to claim 13 where the composition contains:
5% to 30% by weight of 1,2-propyleneglycol;
10% to 20% by weight of glycerin;
5% to 20% by weight of sphingolipid-oil/wax solution;
0.5% to 5% by weight of polyethyleneglycol 75 shea butter glyceride;
10% to 50% by weight of perfluordecalin; and
qs 100% by weight of water,
wherein the sphingolipid is a ceramides, glycosphingolipids or mixtures thereof in a solution of 10% to 20% by weight in an oil or wax solvent.

34. A cosmetic composition according to claim 33 where the oil or wax solvent is a vegetable oil or wax.

35. A cosmetic composition according to claim 34 where the oil or wax solvent comprises jojoba oil.

36. A cosmetic composition according to claim 33 where the composition contains:
15% to 25% by weight of 1,2-propyleneglycol;
14% to 18% by weight of glycerin;
7% to 15% by weight of sphingolipid-oil/wax solution;
1% to 3% by weight of polyethyleneglycol 75 shea butter glyceride;
15% to 25% by weight of perfluordecalin; and
qs 100% by weight of water.
